(19) United States
(12) Patent Application Publication
(10) Pub. No.: US 2020/0301030 A1
(43) Pub. Date: Sep. 24, 2020

(54) SHORT LEADING AND TRAILING FRAMES TO IMPROVE IMAGE QUALITY IN POSITRON EMISSION TOMOGRPAHY (PET)

(71) Applicant: KONINKLIJKE PHILIPS N.V., EINDHOVEN (NL)

(72) Inventors: Andriy ANDREYEV, WILLOUGHBY HILLS, OH (US); Chuanyong BAI, SOLON, OH (US); Xiyun SONG, CUPERTINO, CA (US); Jinghan YE, LIVERMORE, CA (US)

(21) Appl. No.: 16/763,457

(22) PCT Filed: Nov. 6, 2018
(86) PCT No.: PCT/EP2018/080216

§ 371 Date: May 12, 2020

Related U.S. Application Data
(60) Provisional application No. 62/586,229, filed on Nov. 15, 2017.

(51) Int. Cl. G01T 1/29; A61B 6/00; A61B 6/03

(52) U.S. Cl. CPC G01T 1/2985; A61B 6/037; A61B 6/488

(57) ABSTRACT

A non-transitory computer-readable medium storing instructions readable and executable by a workstation (18) including at least one electronic processor (20) to perform an image reconstruction method (100). The method includes: receiving a surview image of a target anatomy acquired by an image acquisition device (12); selecting an axial field of view (FOV) of an imaging volume using the received surview image; optimizing axial boundaries of leading and trailing frames respective to the selected axial FOV of the imaging volume; operating a positron emission tomography (PET) imaging device (14) to acquire PET imaging data including acquiring PET imaging data for the leading frame followed by acquiring PET imaging data for the imaging volume followed by acquiring PET imaging data for the trailing frame; and reconstructing the PET imaging data to generate an image of the selected imaging volume including out-of-axial FOV scatter correction performed using the PET imaging data acquired for at least one of the leading and trailing frames.

### FIELD

The following relates generally to the medical imaging arts, medical image interpretation arts, image reconstruction arts, and related arts.

### BACKGROUND

Positron Emission Tomography (PET) scanners typically have relatively small axial fields-of-view (AFOV) for cost-saving reasons. When a PET scan covers only a portion of the patient body between the head and feet, the acquired data is essentially “truncated” in the axial direction, leading to compromised quality and quantification at and near the first and last axial slices of the image. For example, a cardiac scan may be done using a single frame.

However, in the case of existing hybrid PET/computed tomography (PET/CT) scanners with an AFOV coverage of only 15 cm, the edge slices appear much noisier than the central slices, due to reduced geometric sensitivity in those areas. The effective useful AFOV is thus further reduced. Also, due to the limited AFOV, scatters from the activities in a target portion of the patient, such as the liver, cannot be accurately modelled.

As the market trends towards cost-effective PET/CT, adding more detector elements to the PET/CT scanner is not always a cost-effective way in order to expand the axial sensitivity coverage.

The following discloses new and improved systems and methods to overcome these problems.

### SUMMARY

In one disclosed aspect, a non-transitory computer-readable medium storing instructions readable and executable by a workstation including at least one electronic processor to perform an image reconstruction method. The method includes: receiving a surview image of a target anatomy acquired by an image acquisition device; selecting an axial field of view (FOV) of an imaging volume using the received surview image; optimizing axial boundaries of leading and trailing frames respective to the selected axial FOV of the imaging volume; operating a positron emission tomography (PET) imaging device to acquire PET imaging data including acquiring PET imaging data for the leading frame followed by acquiring PET imaging data for the imaging volume followed by acquiring PET imaging data for the trailing frame; and reconstructing the PET imaging data to generate an image of the selected imaging volume including out-of-axial FOV scatter correction performed using the PET imaging data acquired for at least one of the leading and trailing frames.

In another disclosed aspect, an imaging system includes an image acquisition device configured to acquire a surview image of a target anatomy, and a positron emission tomography (PET) imaging device configured to acquire PET imaging data. At least one electronic processor is programmed to: receive the surview image of the target anatomy; select an axial field of view (FOV) of an imaging volume using the received surview image; optimize overlaps and frame acquisition times of one of the leading and trailing frames respective to the axial FOV of the imaging volume; control the PET imaging device to acquire PET imaging data for the leading frame followed by acquiring PET imaging data for the imaging volume followed by acquiring PET imaging data for the trailing volume; and reconstruct the PET imaging data to generate an image of the imaging volume. The overlaps and frame acquisition times of the leading and trailing frames are optimized to obtain a target sensitivity for the PET imaging data at edge slices located at the overlaps of the axial FOV.

In another disclosed aspect, an imaging system includes an image acquisition device configured to acquire a surview image of a target anatomy, and a positron emission tomography (PET) imaging device configured to acquire PET imaging data. At least one electronic processor is programmed to: receive the surview image of the target anatomy; select an axial field of view (FOV) of an imaging volume using the received surview image; optimize an overlap of a leading frame respective to the axial FOV of the imaging volume; optimize an overlap of a trailing frame respective to the axial FOV of the imaging volume; control the PET imaging device to acquire PET imaging data for the leading frame followed by acquiring PET imaging data for the imaging volume followed by acquiring PET imaging data for the trailing volume; and reconstruct the PET imaging data to generate an image of the imaging volume having a true axial FOV comprising the selected axial FOV, the optimized overlap of the leading frame, and the optimized overlap of the trailing frame. The overlap of the leading frame are optimized separately from and independently of the optimizing of the overlap of the trailing frame.

One advantage resides in providing a cost-effective imaging device.

Another advantage resides in providing an imaging device or method that produces improved overall image quality.

Another advantage resides in providing an imaging device or method that provides better coverage of edge slices (or, said another way, that provides improved sensitivity for the edge slices).

Another advantage resides in providing an imaging device or method with an extended axial field of view.

Another advantage resides in providing an imaging device or method with more accurate scatter correction.

Another advantage resides in providing an imaging device or method that includes leading and trailing image frames designed to reduce an overall number of frames.

Another advantage resides in providing an imaging device or method that includes leading and trailing image frames designed to reduce scanning times.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows image reconstruction system according to one aspect.

FIG. 2 shows an exemplary flow chart operation of the system of FIG. 1;

FIG. 3 illustratively shows an example operation of the system of FIG. 1;

FIG. 4 illustratively shows a graph comparing sensitivities for image studies acquired by the image reconstruction system of FIG. 1.

### DETAILED DESCRIPTION

This disclosure relates to improvements in PET imaging employing one or more axial frames. In an illustrative contemplated implementation, cardiac PET imaging is performed using a PET/CT with a single frame with a system axial extent of approximately 15 cm. In practice, it is found that edge effects at the axial ends of this FOV can lead to the outer edge axial slices being degraded or unusable, leading to an effective axial width of around 12 cm. This is barely large enough to encompass a normal heart, and may truncate the heart if the heart is larger than normal or if the axial positioning of the heart in the PET scanner is less than perfectly centered in the FOV along the axial direction. The degradation of the edge slices has two causes: (1) scattering from out-of-FOV, and (2) low sensitivity (where sensitivity for an edge slice can be viewed as a ratio of total counts for the edge slice compared with total counts for an axial slice located at the center of the FOV).

A known way to compensate for scatter from out-of-FOV is to acquire additional leading and trailing frames that overlap the ends of the single frame FOV by standard 30%-50% and same acquisition time per frame. This extra data enables accurate estimation and correction of the scatter, and increases the sensitivity when data collected in the leading and trailing frames is added to the respective proximate edge slices. However, acquiring the leading and trailing frames increases imaging data collection time.

In improvements disclosed herein, in simulations it has been found that substantial image improvement is obtained by way of even small increases in sensitivity of the edge slices. A sensitivity increase of as low as 5% is sufficient to make the edge slices useable, thereby recovering the entire 15 cm design-basis FOV of the foregoing example. Based on this, it is recognized herein that the acquisition time for the leading and trailing frames can be greatly reduced, e.g. to a few percent of the acquisition time of the imaging frame, so that workflow is not unduly delayed by acquisition of the leading and trailing frames.

For scatter correction, the amount of overlap of the leading and trailing frames can each be optimized for the image protocol. For example, in the case of cardiac PET, the trailing frame captures the liver, which usually accumulates a high concentration of radiopharmaceutical and hence is a substantial source for out-of-FOV scatter. In this case the trailing frame needs to extend axially far enough outside of the FOV to capture most or all of the liver, meaning that the overlap of the trailing frame with the imaging frame is small, e.g. 30% in one example. On the other hand, there is no anatomical structure tending to accumulate high radiopharmaceutical concentration above the heart, and so the leading frame can be constructed to have large overlap with the imaging frame or volume.

With these observations, the following discloses an optimization process for enhancing the value of the leading and trailing frames while minimizing their impact on the workflow. In one approach, an initial CT surview is acquired and the imaging frame positioned based on the cardiac contour observed in the CT surview image. Next, the axial extent and/or overlap of each of the leading and trailing frames is optimized. This may be done using a look-up table that lists the percentage overlap and/or axial extent for these frames for each procedure (e.g. cardiac PET procedure). Additionally or alternatively, if the liver or other out-of-FOV scatter source is discernable in the CT surview image, then a graphical user interface (GUI) may be provided via which the user can manually mark the desired axial extent of the leading and/or trailing frames. Next, the acquisition time for each of the leading frame and the trailing frame is determined based on the chosen axial extent and a threshold sensitivity for the edge slices, e.g. the acquisition time may be chosen to provide a 5% sensitivity at the edge slices.

In some examples, the acquisition time for the imaging frame may be reduced to compensate for the acquisition times of the leading and trailing frames. This effectively trades off image quality to ensure no impact on workflow time.

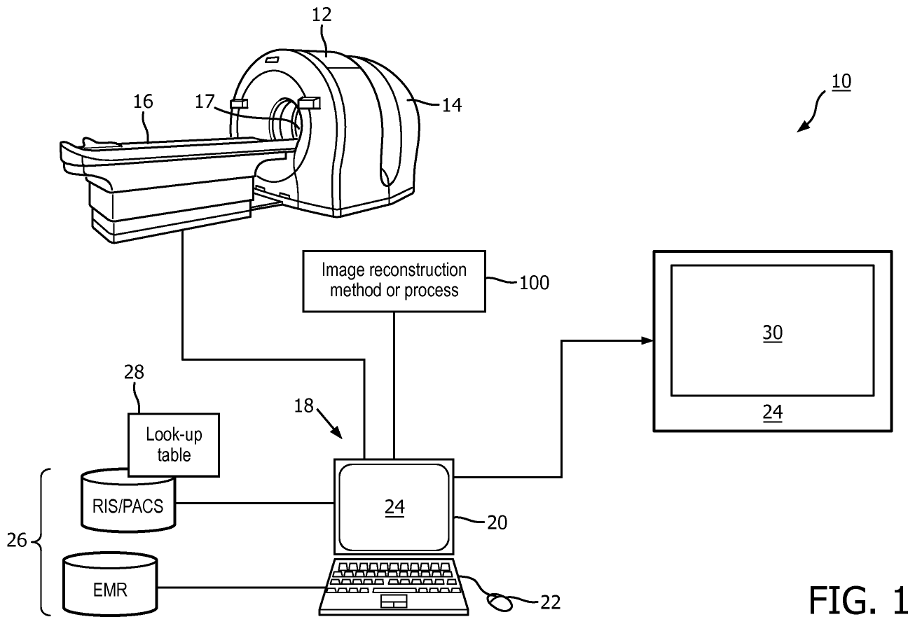

With reference to FIG. 1, an illustrative medical imaging system 10 is shown. As shown in FIG. 1, the system 10 includes an image acquisition device 12. In one example, the image acquisition device 12 can comprise a computed tomography (CT) device. In other examples, the image acquisition device 12 can be any other suitable image acquisition device (e.g., magnetic resonance (MR) devices). The imaging system 10 also includes an emission imaging device 14 (e.g., a positron emission tomography (PET) device or a single photon emission computed tomography (SPECT) device). As referred to herein, the emission imaging device 14 is a PET imaging device (or, in some examples, a time of flight (TOF) PET imaging device). A patient table 16 is arranged to load a patient into an examination region 17, and more particularly can move a prone or supine patient axially either into the examination region of the CT scanner 12 for CT imaging, or into the examination region of the PET scanner 14 for PET imaging. In the illustrative example, the image acquisition device 12 and the PET imaging device 14 are thus combined as a hybrid PET/CT device or a TOF PET/MR device.

The system 10 also includes a computer or workstation or other electronic data processing device 18 with typical components, such as at least one electronic processor 20, at least one user input device (e.g., a mouse, a keyboard, a trackball, and/or the like) 22, and a display device 24. In some embodiments, the display device 24 can be a separate component from the computer 18. The workstation 18 can also include one or more non-transitory storage media 26 (such as a magnetic disk, RAID, or other magnetic storage medium; a solid state drive, flash drive, electronically erasable read-only memory (EEROM) or other electronic memory; an optical disk or other optical storage; various combinations thereof; or so forth), that store one or more databases (e.g., an electronic medical record (EMR) database, a Radiology Information System (RIS) and/or Picture Archiving and Communication System (PACS) database, and the like). The display device 24 is configured to display a graphical user interface (GUI) 30 including one or more fields to receive a user input from the user input device 22.

The at least one electronic processor 20 is operatively connected with the one or more non-transitory storage media 26 which further stores instructions which are readable and executable by the at least one electronic processor 20 to perform disclosed operations including performing an image reconstruction method or process 100. In some examples, the image reconstruction method or process 100 may be performed at least in part by cloud processing.

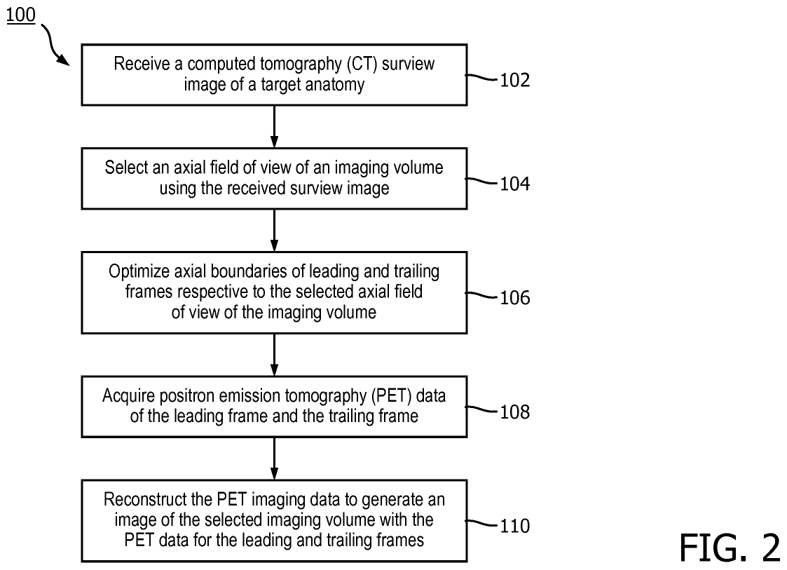

With reference to FIG. 2, an illustrative embodiment of the image reconstruction method 100 is diagrammatically shown as a flowchart. To begin the process, the image acquisition device 12 (e.g., the CT imaging device) is configured, or controlled by the at least one electronic processor 20, to obtain a surview image of a target anatomy (e.g., the heart). At 102, the at least one electronic processor 20 is programmed to receive the surview image of the heart acquired by the CT imaging device 12.

At 104, the at least one electronic processor 20 is programmed to select an axial field of view (AFOV) (also referred to as a scanned axial extent) of an imaging volume using the received surview image. For example, the at least one electronic processor 20 may be programmed to automatically select the AFOV. In other embodiments, the surview image can be displayed on the display device 24, and a medical professional (e.g., a doctor, a technologist, and the like) may select the AFOV via inputs from the user input device 22 (e.g., a mouse click, a keyboard key stroke, and the like) on the GUI 30.

At 106, the at least one electronic processor 20 is programmed to optimize overlapping areas (also referred to herein as overlaps) of leading and trailing frames respective to the selected axial FOV of the imaging volume. In one example, when the surview image is displayed on the display device 24, the at least one electronic processor 20 is programmed to receive at least one user input (e.g., mouse clicks, keystrokes and the like) from the user via the user input device 22. The user inputs are indicative of a selection of one or more overlaps for each of the leading frame and the trailing frame. In another example, the at least one electronic processor 20 is programmed to optimize the overlap of the leading frame and the overlap of the trailing frame with the AFOV of the imaging volume. The overlap of the trailing frame and the overlap of the leading frame may, for example, be selected using a look up table 28 listing at least overlap values for different PET imaging procedures (e.g., cardiac PET, brain PET, and the like). The look up table can be stored in the non-transitory storage medium 26. In some embodiments, both overlaps of each of the leading frame and the trailing frame are optimized. This optimization can be performed by, for example, dragging an outer axial boundary or end of the leading frame and/or the trailing frame in the user interface. In other embodiments, one of the overlaps of either the leading frame or the trailing frame is optimized, while the other overlap of either the leading frame or the trailing frame is set to a fixed factory-optimized value. In some embodiments, both overlaps of each of the leading and trailing frames are optimized respective to the AFOV of the imaging volume separately and independently of each other.

At 108, the at least one electronic processor 20 is programmed to operate the PET imaging device 14 to acquire PET imaging data including acquiring PET imaging data for the leading frame followed by acquiring PET imaging data for the imaging volume followed by acquiring PET imaging data for the trailing frame.

In other embodiments, the at least one electronic processor 20 is programmed to optimize the overlaps of leading and trailing frames respective to objectives including maximizing coverage of at least one region physiologically predisposed to accumulate radiopharmaceutical used in the acquisition of the PET imaging data. For example, in the case of cardiac PET the target anatomy shown in the surview image is the heart, at least one of the regions physiologically predisposed to accumulate the type(s) of radiopharmaceutical typically used in the acquisition of the cardiac PET imaging data is the liver. The overlaps of the leading or the trailing frame (whichever is disposed below the heart and hence overlapping the liver) are therefore optimized to encompass the liver. In a manual approach, the surview CT image is displayed on the display 24. As at least the outline of the heart and liver are generally visible in CT, the user can see both the heart and the liver in the displayed surview image in order to be able to operate the GUI 30 to set the overlaps of the proximate leading or trailing frame to encompass the liver. Alternatively, in an automated approach the surview CT image is automatically segmented to identify the liver overlap and the overlaps of the proximate leading or trailing frame are automatically set to encompass the segmented liver area.

Additionally or alternatively, another objective of the optimization of the leading and trailing frames can include determining the overlaps and/or acquisition times of the leading and trailing frames so as to provide a target sensitivity for the PET imaging data at edge slices located at axial boundaries of the AFOV. In some examples, the overlaps of the leading and trailing frames are optimized to provide the target sensitivity for the edge slices. In other examples, the frame acquisition times of the PET imaging data for the leading and trailing frames are optimized to provide the target sensitivity increase for the edge slices of the AFOV. Based on simulations, it is expected that a target sensitivity increase of at least 5% of the peak value is sufficient to allow the edge slices to be usable for diagnostic purposes, ensuring the full AFOV is clinically useful. Based on this relatively low sensitivity increase of 5% or higher, it is recognized herein that the acquisition time for the leading and trailing frames can be greatly reduced, e.g. to a few percent of the acquisition time of the imaging frame, while still providing sufficient “extra” data at the edge slices to ensure usable sensitivity is achieved, so that workflow is not unduly delayed by acquisition of the leading and trailing frames.

In some examples, the overall acquisition time can be expressed according to Equation 1:

T_{O}=T_{L}+T_{P}+T_{T} Equation (1)

where the T_{O} is the overall acquisition time, T_{L} is the acquisition time for the leading frame, T_{P} is the acquisition time for a current frame, T_{T} is the acquisition time for the trailing frame. When there is a restriction on the overall acquisition time T_{O} increase, then the addition of the non-zero T_{L} and T_{T} leads to decrease of the T_{P} as compensation as well as small time required to shift the patient table into new scanning position.

The amount of the axial overlap between the leading and the current frame O_{L}, as well as the amount of axial overlap between the trailing and current frame O_{T} also determines the amount of counts C_{P} that are detected during acquisition in primary volume of interest determined by current imaging frame, as expressed in Equation 2:

C_{P}=S_{L∩P}(O_{L})T_{L}+S_{P}T_{P}+S_{T∩P}(O_{T})T_{T} Equation (2)

where S_{P} is the amount of counts detected in the primary frame per unit time, S_{L∩P}(O_{L}) and S_{T∩P}(O_{T}) is the amount of counts detected in leading and trailing frame that contribute to the primary imaging volume, respectively. Equations 1 and 2 can further used to determine detected counts C_{P}_{ᵢ} in each volume element of the primary imaging frame. The exact expression for C_{P}_{ᵢ} depends on the geometric sensitivity profile, detector efficiency, amount of overlap, with the following optimizable parameters: O_{L}, O_{T}, T_{L}, T_{P} and T_{T}, to achieve the minimum required value for C_{P}_{ᵢ}.

At 110, the at least one electronic processor 20 is programmed to reconstruct the PET imaging data to generate an image of the selected imaging volume including out-of-AFOV scatter correction performed using the PET imaging data acquired from the leading and trailing frames. The data from the short leading and trailing frames are also used in the reconstruction to reduce a noise level near or at the edge slices of the reconstructed frames of interest (that is, the added data from the leading and trailing frames increases sensitivity of the edge slices sufficiently to reduce the noise level to a clinically acceptable level). In some examples, the reconstructed imaging volume can include a true axial FOV comprising the selected axial FOV, the optimized overlap of the leading frame, and the optimized overlap of the trailing frame

As disclosed herein, for scatter correction, the amount of overlap of the leading and trailing frames can each be optimized for the image protocol. For example, in the case of cardiac PET, if the trailing frame captures the liver, which usually accumulates a high concentration of radiopharmaceutical and hence is a substantial source for out-of-FOV scatter, then the trailing frame can be extended axially far enough outside of the FOV to capture most or all of the liver, by reducing the overlap between the primary frame and the trailing frame, e.g. from 50% to 30% in one example. On the other hand, there is no anatomical structure tending to accumulate high radiopharmaceutical concentration above the heart, and so the leading frame in this case can be constructed to have large overlap with the imaging frame. It can also be foreseen that either leading or trailing frame can be fully overlapped with primary frame (thus disappearing from the image acquisition protocol), when there is no need for additional extension of the axial FOV in certain direction.

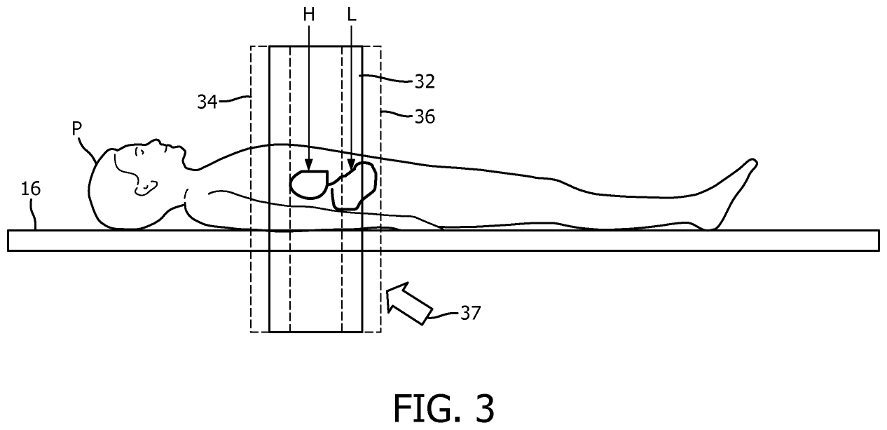

FIG. 3 illustratively shows an example of a display (e.g., on the display 24 of FIG. 1) of the surview image acquired by the CT scanner or other image acquisition device 12. The surview image depicts the patient P on the table or patient support 16. The image of the heart H and liver L are also denoted. As shown in FIG. 3, superimposed on the surview image is a diagrammatic depiction of the boundary of a current or primary frame 32 (shown by a solid line) to be acquired by the PET imaging, along with a diagrammatic depiction of the boundary of the leading frame 34 (i.e., a frame disposed before and overlapping with the current frame), and the boundary of the trailing frame 36 (e.g., a frame overlapping with and disposed after the current frame). The user can use the GUI 30 to adjust the overlaps of the leading and trailing frames 34, 36, e.g. FIG. 3 shows a mouse pointer 37 being used to adjust an overlap of the trailing frame 36 so that the trailing frame encompasses the liver L. In an automated embodiment, automatic segmentation is employed to delineate the boundaries of the heart H and liver L and these automatically determined boundaries are used to set the overlaps of the trailing frame 36.

In some examples, the overlapping of the leading and/or trailing frames with the primary frame is much larger than the typical overlapping, such as 80% overlapping (rather than the industry standard 15%-50%). By using the large overlap, the majority of the events from the leading and trailing frames can directly contribute to the reconstruction of the primary frame. As a result, more time can be spent on selecting the leading/trailing frames to improve the statistics of those frames for more accurate scatter correction or sensitivity, while the majority of the counts are directly used to reconstruct the image of the primary frame.

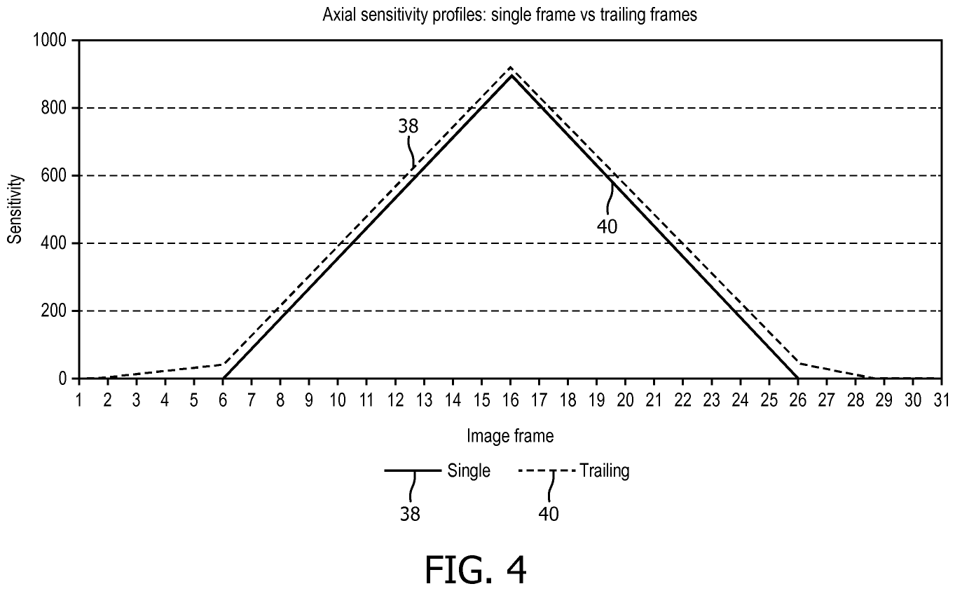

FIG. 4 shows an example of this sensitivity principle. FIG. 4 shows a graph showing a first simulation study 38 including a single standard frame acquisition of 90 seconds, and a second simulation study 40 including a single frame acquisition of 80 seconds with addition of leading and trailing frames of 10 seconds each with 70% frame overlap. FIG. 4 shows the corresponding sensitivities of the first and second studies 38 and 40 as a function of axial frame. The sensitivity for the second study 40 (i.e., including the leading and trailing frames) has a higher sensitivity than the first study 38 (i.e., including only a primary frame). As a result, the total acquisition time is going to increase by only 10 seconds, but with added benefits of better count statistics for edge slices, as well as accurate sample of outside-FOV activity estimation.

In some embodiments, the CT scans acquired by the image acquisition device 12 for an extended portion of the target anatomy covered by leading/trailing frames should not be necessary especially if using TOF non-attenuation-corrected (NAC) reconstruction. This avoids the concern of increased patient dose due to CT over-scan. The TOF NAC or TOF-derived-AC reconstruction restores and activity distribution in the leading and/or trailing frames accurate enough for accurate scatter modeling and correction. The overlapping region of the reconstructed leading frame and primary frame can be used to normalize the leading/trailing frame images to the primary frame image, therefore, the resulted leading/trailing images are sufficient for scatter correction.

In other embodiments, filtering of the images from the leading and/or trailing frames may be necessary to improve the scatter modelling accuracy. The short leading and/or trailing scan images can be scaled up by scanning time to improve the reconstruction of the frame of interest.

The disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A non-transitory computer-readable medium storing instructions readable and executable by a workstation including at least one electronic processor to perform an image reconstruction method, the method comprising:
receiving a surview image of a target anatomy acquired by an image acquisition device;
selecting an axial field of view (FOV) of an imaging volume using the received surview image;
optimizing overlaps of one of the leading and trailing frames respective to the selected axial FOV of the imaging volume;
operating a positron emission tomography (PET) imaging device to acquire PET imaging data including acquiring PET imaging data for the leading frame followed by acquiring PET imaging data for the imaging volume followed by acquiring PET imaging data for the trailing frame; and
reconstructing the PET imaging data to generate an image of the selected imaging volume including out-of-axial FOV scatter correction performed using the PET imaging data acquired from the optimized at least one of the leading and trailing frames.

2. The non-transitory computer-readable medium of claim 1, wherein the imaging volume comprises a plurality of frames including an overlap between each frame, and wherein:
the non-transitory computer-readable medium further stores a look-up table listing overlaps for the leading and trailing frames for different imaging procedures; and
wherein the optimizing includes at least one of:
optimizing the overlap of the leading frame with the axial FOV of the imaging volume wherein the overlap is selected using the look-up table; and
optimizing the overlap of the trailing frame with the axial FOV of the imaging volume wherein the overlap is selected using the look-up table.

3. The non-transitory computer-readable medium of claim 1, wherein the optimizing includes:
operating a display device to present the surview image; and
receiving user inputs via a user input device using a graphical user interface employing the display wherein the user inputs mark at least one overlap for the leading frame and at least one overlap for the trailing frame.

4. The non-transitory computer-readable medium of claim 1, wherein the optimizing includes:
optimizing the overlap for both the leading frame and the overlap for trailing frame.

5. The non-transitory computer-readable medium of claim 1, wherein the optimizing includes:
for the leading frame, optimizing the overlap whereby the trailing frame overlap is fixed to the default value; and
for the trailing frame, optimizing the overlap whereby the leading frame overlap is fixed to the default value.

6. The non-transitory computer-readable medium of claim 1, wherein the optimizing includes:
optimizing the overlap of the leading frame respective to the axial FOV of the imaging volume; and
optimizing the overlap of the trailing frame respective to the axial FOV of the imaging volume;
wherein the overlaps of the leading frame are optimized separately from and independently of the optimizing of the axial boundaries of the trailing frame.

7. The non-transitory computer-readable medium of claim 1, wherein the optimizing includes:
optimizing the overlaps of leading and trailing frames respective to objectives including maximizing the overlap with at least one region physiologically predisposed to accumulate radiopharmaceutical used in the acquisition of the PET imaging data.

8. The non-transitory computer-readable medium of claim 7, wherein the optimizing includes:
optimizing the overlaps of leading and trailing frames respective to objectives including a target sensitivity for the PET imaging data at edge slices located at the axial boundaries of the axial FOV.

9. The non-transitory computer-readable medium of claim 8, further comprising:
optimizing frame acquisition times for acquiring PET imaging data for the leading and trailing frames to obtain a target sensitivity for the PET imaging data at edge slices located at the axial boundaries of the axial FOV.

10. The non-transitory computer-readable medium of claim 8, wherein the target sensitivity increase is at least 5% of the peak value; and
the overlap for the leading and trailing frames is more than 50%.

11. The non-transitory computer-readable medium of claim 1, wherein:
the imaging volume is a cardiac imaging volume; and
the optimizing includes optimizing the overlaps of the leading or trailing frame to encompass a liver.

12. An imaging system, comprising:
an image acquisition device configured to acquire a surview image of a target anatomy;
a positron emission tomography (PET) imaging device configured to acquire PET imaging data; and
at least one electronic processor programmed to:
receive the surview image of the target anatomy;
select an axial field of view (FOV) of an imaging volume using the received surview image;
optimize overlaps and frame acquisition times of one of the leading and trailing frames respective to the axial FOV of the imaging volume;
control the PET imaging device to acquire PET imaging data for the leading frame followed by acquiring PET imaging data for the imaging volume followed by acquiring PET imaging data for the trailing volume; and
reconstruct the PET imaging data to generate an image of the imaging volume;
wherein the overlaps and frame acquisition times of the one of the leading and trailing frames are optimized to obtain a target sensitivity for the PET imaging data at edge slices located at the axial boundaries of the axial FOV.

13. The imaging system of claim 12, wherein the at least one electronic processor is further programmed to:
optimize the overlap of the leading frame with the axial FOV of the imaging volume wherein the overlap is selected using a look-up table listing at least overlap values for different imaging procedures; and
optimize the overlap of the trailing frame with the axial FOV of the imaging volume wherein the overlap is selected using the look-up table.

14. The imaging system of claim 12, wherein the at least one electronic processor is further programmed to:
operate a display device to present the surview image; and
receive user inputs via a user input device using a graphical user interface employing the display wherein the user inputs mark the overlap for the leading frame and the overlap for the trailing frame.

15. The imaging system of claim 12, wherein the at least one electronic processor is further programmed to:
optimizing both of the overlaps for the leading frame and the overlap for trailing frame.

16. The imaging system of claim 12, wherein the at least one electronic processor is further programmed to:
for the leading frame, optimizing the overlap whereby the trailing frame overlap is fixed to the default value; and
for the trailing frame, optimizing the overlap whereby the leading frame overlap is fixed to the default value.

17. The imaging system of claim 12, wherein the at least one electronic processor is further programmed to:
optimize the overlap of the leading frame respective to the axial FOV of the imaging volume; and
optimize the overlap of the trailing frame respective to the axial FOV of the imaging volume;
wherein the overlaps of the leading frame are optimized separately from and independently of the optimizing of the axial boundaries of the trailing frame.

18. The imaging system of claim 12, wherein the at least one electronic processor is further programmed to:
optimize the overlaps of leading and trailing frames respective to objectives including maximizing the overlap with at least one region physiologically predisposed to accumulate radiopharmaceutical used in the acquisition of the PET imaging data.

19. An imaging system, comprising:
an image acquisition device configured to acquire a surview image of a target anatomy;
a positron emission tomography (PET) imaging device configured to acquire PET imaging data; and
at least one electronic processor programmed to:
receive the surview image of the target anatomy;
select an axial field of view (FOV) of an imaging volume using the received surview image;
optimize an overlap of a leading frame respective to the axial FOV of the imaging volume;
optimize an overlap of a trailing frame respective to the axial FOV of the imaging volume;
control the PET imaging device to acquire PET imaging data for the leading frame followed by acquiring PET imaging data for the imaging volume followed by acquiring PET imaging data for the trailing volume; and
reconstruct the PET imaging data to generate an image of the imaging volume having a true axial FOV comprising the selected axial FOV, the optimized overlap of the leading frame, and the optimized overlap of the trailing frame;
wherein the axial boundaries of the leading frame are optimized separately from and independently of the optimizing of the axial boundaries of the trailing frame.

20. The imaging system of claim 19, wherein the at least one electronic processor is further programmed to at least one of:
reconstructing the PET imaging data to generate an image of the imaging volume including out-of-axial FOV scatter correction performed using the PET imaging data acquired for the leading and trailing frames; and
optimizing the overlaps and frame acquisition times of the leading and trailing frames are to obtain a target sensitivity for the PET imaging data at edge slices located at the axial boundaries of the axial FOV.
